(19) United States
(12) Patent Application Publication
(10) Pub. No.: US 2003/0050279 A1
(43) Pub. Date: Mar. 13, 2003

(54) Beta-glucans

(76) Inventors: Federico Federici, Perugia (IT); Maurizio Petruccioli, Viterbo (IT); Peter Van Den Broek, Epalinges (CH); Francesca Stingele, Lausanne (CH)

Correspondence Address: WINSTON & STRAWN, PATENT DEPARTMENT, 1400 L STREET, N.W., WASHINGTON, DC, 20005-3502, US

(21) Appl. No.: 10/236,991

(22) Filed: Sep. 5, 2002

Related U.S. Application Data
(63) Continuation of application No. PCT/EP01/03100, filed on Mar. 20, 2001.

(30) Foreign Application Priority Data
Mar. 24, 2000 (EP) 00106406.2

(51) Int. Cl. A61K 31/715; C12P 19/04

(52) U.S. Cl. 514/54; 435/101

(57) ABSTRACT

A method for producing a beta-glucan from a non-pathogenic saprophytic filamentous fungus or composition that contains it. Also, methods for providing this beta-glucan in a food product to improve structure, texture, stability or combinations thereof, in a food product to provide nutrition or in the manufacture of a medicament or nutritional composition for the prevention or treatment of an immune disorder, tumor or microbial infection.

### CROSS-REFERENCE TO RELATED APPLICATIONS

This application is a continuation of the U.S. National Stage designation of International application no. PCT/EP01/03100 Filed Mar. 20, 2001, the entire content of which is expressly incorporated herein by reference thereto.

### TECHNICAL FIELD

The present invention relates to a method of producing a beta-glucan; use of a non-pathogenic saprophytic filamentous fungus or composition comprising it for providing a beta-glucan and thereby improving food structure, texture, stability or a combination thereof; use of a non-pathogenic saprophytic filamentous fungus for providing a beta-glucan and thereby providing nutrition; and use of a fungus or composition comprising it in the manufacture of a medicament or nutritional composition for the prevention or treatment of an immune disorder, tumor or microbial infection.

### BACKGROUND ART

Over the last decade there has been a great deal of interest in biopolymers from microbial origins in order to replace traditional plant—and animal derived gums in nutritional compositions. New biopolymers could lead to the development of materials with novel, desirable characteristics that could be more easily produced and purified. For this reason, the characterization of exopolysaccharide (“EPS”) production at a biochemical as well as at a genetic level has been studied. An advantage of EPS is that it can be secreted by food micro-organisms during fermentation, but using EPS produced by micro-organisms gives rise to the problem that the level of production is very low (50-500 mg/l) and that once the EPS is extracted it loses its texturing properties.

One example of an EPS is a beta-glucan. Beta-glucans are made of a β-glucose which are linked by 1-3 or 1-6 bonds and have the following characteristics that are attractive to processors in the food-industry: viscosifying, emulsifying, stabilising, cryoprotectant and immune-stimulating activities.

Remarkably, it has been found that fungi can produce high amounts of biopolymers (20 g/l) such as beta-glucans. One example is scleroglucan, a polysaccharide produced by certain filamentous fungi (e.g. Sclerotinia, Corticium, and Stromatina species) which, because of its physical characteristics, has been used as a lubricant and as a pressure-compensating material in oil drilling (Wang, Y., and B. Mc Neil. 1996. Scleroglucan. Critical Reviews in Biotechnology 16: 185-215).

Scleroglucan consists of a β(1-3) linked glucose backbone with different degrees of β(1-6) glucose side groups. The presence of these side groups increases the solubility and prevents triple helix formation that, by consequence, decreases its ability to form gels. The viscosity of scleroglucan solutions shows high tolerance to pH (pH 1-11), temperature (constant between 10-90° C.) and electrolyte change (e.g. 5% NaCl, 5% CaCl₂). Furthermore, its applications in the food industry for bodying, suspending, coating and gelling agents have been suggested and strong immune stimulatory, anti-tumor and anti-microbial activities have been reported (Kulicke, W. M., A. I. Lettau, and H. Thielking. 1997, Correlation between immunological activity, molar mass, and molecular structure of different (1→3)-β-D-glucans. Carbohydr. Res. 297: 135-143).

As there is a need for these type materials in the food industry, they have been further investigated by the present inventors, and this invention now has identified unexpected benefits in food processing operations due to the use of these materials.

### SUMMARY LF THE INVENTION

Remarkably, a class of filamentous fungi has now been identified and isolated which has been found to produce a fungal exopolysaccharide that exhibits characteristics that are attractive to the food industry. Two aspects of the EPS of interest are (a) its good texturing properties and (b) its ability to promote an immuno-stimulatory effect in in vitro and in vivo immunological assays. The fungal EPS could be incorporated into a health food (e.g., EPS as texturing fat replacer for low-calorie products or new immuno-stimulatory products) or provided alone for example as a food supplement.

Surprisingly, it has also been found that these fungi are able to produce a remarkably high yield of a beta-glucan.

Accordingly, in a first aspect the present invention provides a method of producing a beta-glucan which comprises fermenting a suspension comprising a non-pathogenic saprophytic filamentous fungus under conditions sufficient to produce a beta-glucan and extracting a beta-glucan from the fermented suspension.

In a second aspect the present invention provides a method of enhancing one or more of structure, texture, or stability of a food product which comprises providing a beta-glucan by a non-pathogenic saprophytic filamentous fungus or composition containing same, and adding the beta-glucan to the food product in an amount effective to thereby enhance food structure, texture, stability or combinations thereof.

In another aspect, the invention relates to a method of providing nutrition in a food product which comprises providing a beta-glucan by a non-pathogenic saprophytic filamentous fungus or composition containing same, and adding the beta-glucan to the food product in an amount sufficient to increase its nutrition content.

Yet another aspect of the invention relates to a method for manufacturing a medicament or nutritional composition for the prevention or treatment of an immune disorder, tumor or microbial infection which comprises providing a beta-glucan by a non-pathogenic saprophytic filamentous fungus or composition containing same, and forming a medicament or nutritional composition from a therapeutically effective amount of the beta-glucan.

In these methods of use the beta-glucan can be provided by the production methods described herein

### DETAILED DESCRIPTION OF THE PREFERRED EMBODIMENTS

One or more of a non-pathogenic saprophytic filamentous fungus selected from the group consisting of Penicillium chermesinum, Penicillium ochrochloron, Rhizoctonia sp., Phorna sp., and combinations thereof is fermented to form the beta-glucan. Preferably, at least three of these fungi are fermented together. More preferably all of these fungi are fermented together.

The fermenting step is conducted for at least about 50 hours, preferably for about 80 hours to about 120 hours, and even more preferably for about 96 hours. These times are advantageous for obtaining high yields of beta-glucan.

The fermenting step is advantageously conducted in suspension in a medium comprising at least one component selected from the group consisting of NaNO₃, KH₂PO₄, MgSO₄, KCl and yeast extract. Preferably, at least two or three of these components are used and most preferably all these components are used together to provide the best yields of beta-glucan. Advantageously, the beta-glucan is added to a food product, a nutritional composition, or a medicament.

Preferably, the fungus is cultivated in a minimal medium. More preferably, the medium consists essentially of glucose and salts, and provides the advantage of enabling isolation of a highly pure polysaccharide at the expense of the production yield. This is because yeast extract contains polysaccharides that are difficult to separate from the EPS. Most preferably, the medium comprises NaNO₃ (10 mM), KH₂PO₄ (1.5 g/l), MgSO₄ (0.5 g/l), KCl (0.5), C₄H₁₂N₂O₆ (10 mM) glucos (60) and has a pH of 4.7.

The suitable fungus that can be used according to the invention includes those selected from the group consisting of Penicillium chermesinum, Penicillium ochrochloron, Rhizoctonia sp., Phoma sp., or a combination thereof.

Additional features and advantages of the present invention are described in, and will be apparent from the description of the most preferred embodiments which are set out below and in the examples.

In one preferred embodiment, beta-glucans are produced by fermenting a suspension which comprises a fungus in a medium of (g/l) NaNO₃ (3), KH₂PO₄ (1), MgSO₄ (0.5), KCl (0.5), Yeast Extract (1.0), and glucose (30) with the pH of medium adjusted to 4.7. The fermentation is allowed to proceed for about 96 hours at about 28° C. with shaking at about 18 rpm. In an alternative embodiment, strains which initially do not appear to produce the polysaccharide are incubated for about 168 hours and then are added to the medium under the previously described conditions.

### EXAMPLES

The following examples are given by way of illustration only and in no way should be construed as limiting the subject matter of the present application.

### Example 1

### Fungal Beta-Glucan Production

The following fungal isolates were isolated and classified:

| Lab-isolate | “Italian”, public name | CBS identification |
|---|---|---|
| P28 | Penicillium chermesinum | Penicillium glabrum |
| | | (teleomorph*) |
| P45 | Penicillium ochrochloron | Eupenicillium euglaucum |
| | | (anamorph**) |
| P82 | Rhizoctonia sp. | Botryosphaeria rhodina |
| | | (teleomorph)/ |
| | | Lasiodiplodia theobromae |
| | | (anamorph) |
| P98 | Phoma sp. | N/A |
| VT13 | Phoma sp. | N/A |
| VT14 | Phoma sp. | N/A |

**anamorph = asexual form, *teleomorph = sexual form
N/A = not available.

### Example 2

### Standard Polysaccharide Production

Media TB1 (g/l) was used as follows: NaNO₃ (3), KH₂PO₄ (1), MgSO₄ (0.5), KCl (0.5), Yeast Extract (1.0), and glucose (30) with the pH adjusted to 4.7.

The fermentation time was 96 h at 28° C. with shaking at 180 rpm. For strains which initially did not seem to produce any polysaccharide the incubation was prolonged to 168 h.

Results of polysaccharide production were as follows:

| | | | | Specific |
|---|---|---|---|---|
| | Biomass | Polysaccharide | | production |
| Fungal strain | (g/l) | (g/l) | pH | (g/g) |
| Slerotium glucanicum NRRL 3006 | 9.06 ± 2.06 | 11.20 ± 0.71 | 3.79 | 1.24 |
| Botritis cinerea P3 | 2.64 ± 0.10 | 5.90 ± 0.57 | 4.35 | 2.23 |
| Sclerotinia sclerotiorum P4 | 1.16 ± 0.16 | 1.61 ± 0.13 | 2.50 | 1.38 |
| Fusarium culmorum P8 | 6.51 ± 1.05 | 0.82 ± 0.13 | 7.70 | 0.13 |
| Not identified P9 | 5.43 ± 0.53 | 1.32 ± 0.02 | 4.00 | 0.24 |
| Penicillium chermesinum P28 | 4.08 ± 1.17 | 0.68 ± 0.11 | 3.30 | 0.17 |
| Penicillium ochrochloron P45 | 10.53 ± 2.87 | 0.45 ± 0.07 | 3.50 | 0.04 |
| Fusarium sp. P58 | 8.60 ± 2.12 | 1.25 ± 0.35 | 7.44 | 0.15 |
| Sclerotinia sclerotiorum P62 | 2.10 ± 0.00 | 0.86 ± 0.00 | 3.80 | 0.41 |
| Sclerotinia sclerotiorum P63 | 4.08 ± 0.54 | 1.33 ± 0.04 | 3.30 | 0.33 |
| Botritis fabae P65 | 19.70 ± 0.00 | 0.50 ± 0.00 | 4.94 | 0.03 |
| Rhizoctonia fragariae P70 | 12.52 ± 0.40 | 1.55 ± 0.07 | 8.60 | 0.12 |
| Colletotrichum acutatum P72 | 6.01 ± 0.89 | 1.05 ± 0.07 | 7.00 | 0.17 |
| Pestalotia sp. P75 | 8.70 ± 0.28 | 1.90 ± 0.28 | 6.30 | 0.22 |
| Colletotrichum sp. P80 | 12.00 ± 1.95 | 0.65 ± 0.07 | 6.50 | 0.05 |
| Colletotrichum sp. P81 | 5.10 ± 0.71 | 0.80 ± 0.00 | 5.70 | 0.16 |
| Rhizoctonia sp. P82 | 5.70 ± 0.28 | 8.90 ± 1.56 | 6.50 | 1.56 |
| Acremonium sp. P83 | 4.69 ± 0.62 | 1.45 ± 0.07 | 7.20 | 0.31 |
| Acremonium sp. P84 | 5.50 ± 0.00 | 1.30 ± 0.00 | 7.20 | 0.24 |
| Acremonium sp. P86 | 3.90 ± 0.71 | 1.00 ± 0.14 | 5.85 | 0.26 |
| Acremonium sp. P90 | 8.08 ± 0.01 | 0.73 ± 0.18 | 4.40 | 0.09 |
| Not identified P91 | 10.50 ± 0.14 | 1.28 ± 0.31 | 6.83 | 0.12 |
| Chaetomium sp. P94 | 8.30 ± 1.43 | 1.00 ± 0.28 | 7.40 | 0.12 |
| Phoma herbarum P97 | 13.61 ± 2.34 | 0.98 ± 0.22 | 7.50 | 0.07 |
| Phoma sp. P98 | 11.01 ± 1.07 | 2.89 ± 0.01 | 8.00 | 0.26 |
| Phoma sp. P99 | 11.76 ± 1.66 | 0.66 ± 0.04 | 6.45 | 0.06 |

*Values are given at the time of maximum EPS production. Data are means of two independent experiments ± standard deviation.

### Example 3

### Optimized Polysaccharde Production

Polysaccharide production by Rhizoctonia sp. P82, Phoma sp. P98 and Penicillium chermesinum P28 were studied. The results were as follows:

A. Effect of carbon source cultivated on TB1:

I. EPS production by Rhizoctonia sp. P82

| Carbon | Biomass | Polysaccharide | | Specific production |
|---|---|---|---|---|
| source** | (g/l) | (g/l) | pH | (g/g) |
| Glucose | 3.74 ± 0.80 | 18.55 ± 0.57 | 5.48 | 4.96 |
| Fructose | 4.20 ± 0.58 | 21.10 ± 0.89 | 5.60 | 5.02 |
| Galactose | 4.21 ± 0.19 | 16.67 ± 1.20 | 6.52 | 3.96 |
| Xylose | 3.45 ± 0.53 | 15.94 ± 2.42 | 6.07 | 4.63 |
| Sorbitol | 5.19 ± 0.80 | 4.70 ± 0.21 | 6.16 | 0.91 |
| Glycerol | 5.25 ± 0.60 | 1.54 ± 0.42 | 6.15 | 0.29 |
| Sucrose | 4.03 ± 0.59 | 14.07 ± 0.64 | 5.61 | 3.49 |
| Maltose | 4.07 ± 0.32 | 12.22 ± 0.34 | 5.28 | 3.00 |
| Lactose | 4.63 ± 0.47 | 8.78 ± 0.59 | 6.34 | 1.90 |
| Starch | 5.77 ± 0.95 | 17.36 ± 0.69 | 6.26 | 3.01 |

*Values are given at the time of maximum EPS production. Data are means of three independent experiments ± standard deviation.
**Carbon sources were added to the medium at 30 g/l.

II. EPS production by Phoma sp. P98.

| Carbon | Biomass | Polysaccharide | | Specific production |
|---|---|---|---|---|
| source** | (g/l) | (g/l) | PH | (g/g) |
| Glucose | 11.99 ± 0.64 | 1.97 ± 1.22 | 7.31 | 0.16 |
| Fructose | 11.11 ± 0.76 | 1.22 ± 0.45 | 7.35 | 0.11 |
| Galactose | 10.35 ± 0.78 | 4.12 ± 0.03 | 7.44 | 0.40 |
| Xylose | 11.47 ± 1.40 | 2.57 ± 0.27 | 7.35 | 0.22 |
| Sorbitol | 11.17 ± 0.69 | 7.54 ± 1.10 | 7.10 | 0.68 |
| Glycerol | 11.00 ± 0.37 | 0.63 ± 0.05 | 7.29 | 0.06 |
| Sucrose | 12.93 ± 0.44 | 2.91 ± 0.55 | 7.36 | 0.23 |
| Maltose | 12.50 ± 0.18 | 2.65 ± 0.98 | 6.92 | 0.21 |
| Lactose | 9.77 ± 0.01 | 1.06 ± 0.14 | 7.05 | 0.11 |
| Starch | 13.51 ± 1.65 | 2.28 ± 0.11 | 7.43 | 0.17 |

*Values are given at the time of maximum EPS production. Data are means of three independent experiments ± standard deviation.
**Carbon sources were added to the medium at 30 g/l.

III. EPS production by Penicillium chermesinum P28*.

| Carbon | Biomass | Polysaccharide | | Specific production |
|---|---|---|---|---|
| source** | (g/l) | (g/l) | PH | (g/g) |
| Glucose | 11.69 ± 0.04 | 0.59 ± 0.13 | 3.51 | 0.05 |
| Fructose | 12.91 ± 1.20 | 0.46 ± 0.06 | 3:64 | 0.04 |
| Galactose | 8.64 ± 2.09 | 0.00 ± 0.00 | 5.23 | 0.00 |
| Xylose | 10.68 ± 0.06 | 0.41 ± 0.13 | 3.57 | 0.04 |
| Sorbitol | 8.58 ± 1.67 | 1.09 ± 0.01 | 5.07 | 0.13 |
| Glycerol | 13.06 ± 1.05 | 0.18 ± 0.04 | 3.57 | 0.01 |
| Sucrose | 13.11 ± 0.80 | 0.59 ± 0.11 | 3.44 | 0.05 |
| Maltose | 10.90 ± 1.11 | 0.61 ± 0.16 | 3.53 | 0.06 |
| Lactose | 9.38 ± 0.34 | 0.00 ± 0.00 | 4.69 | 0.00 |
| Starch | 9.92 ± 2.04 | 0.50 ± 0.05 | 3.58 | 0.05 |

*Values are given at the time of maximum EPS production. Data are means of three independent experiments ± standard deviation.
**Carbon sources were added to the medium at 30 g/l.

B. Effect of glucose concentration cultivated on TB 1:

I. EPS production by Rhizoctonia sp. P82*.

| Glucose | Biomass | Polysaccharide | | Specific production |
|---|---|---|---|---|
| (g/l) | (g/l) | (g/l) | pH | (g/g) |
| 30 | 3.74 ± 0.80 | 18.55 ± 0.57 | 5.85 | 4.96 |
| 40 | 7.29 ± 0.42 | 21.40 ± 0.89 | 6.03 | 2.94 |
| 50 | 8.30 ± 0.74 | 30.20 ± 1.47 | 5.67 | 3.64 |
| 60 | 8.17 ± 1.34 | 35.26 ± 1.64 | 6.13 | 4.32 |

*Values are given at the time of maximum EPS production. Data are means of three independent experiments ± standard deviation.

II. EPS production by Phoma sp. P98*.

| Sorbitol | Biomass | Polysaccharide | | Specific production |
|---|---|---|---|---|
| (g/l) | (g/l) | (g/l) | pH | (g/g) |
| 30 | 8.60 ± 0.88 | 5.78 ± 0.61 | 7.22 | 0.67 |
| 40 | 12.08 ± 0.71 | 8.76 ± 0.40 | 7.12 | 0.73 |
| 50 | 13.22 ± 1.43 | 10.70 ± 0.48 | 7.13 | 0.81 |
| 60 | 16.47 ± 0.21 | 13.11 ± 0.33 | 7.56 | 0.80 |

*Values are given at the time of maximum EPS production. Data are means of three independent experiments ± standard deviation.

Surprisingly, it can be seen from the results that increasing the concentration of the carbon source (glucose and sorbitol for Rhizoctonia sp. P82 and Phonza sp. P98, respectively), EPS production by both strains increased markedly (approx. 100% increase) reaching a maximum of 35.2 and 13.1 g/l, respectively.

C. Effect of nitrogen source cultivated on TB 1:

I. EPS production by Rhizoctonia sp. P82.*

| Nitrogen | Biomass | Polysaccharide | | Specific production |
|---|---|---|---|---|
| source | (g/l) | (g/l) | PH | (g/g) |
| NaNO₃ | 3.74 ± 0.80 | 18.55 ± 0.57 | 5.53 | 4.96 |
| NH₄NO₃ | 4.05 ± 0.29 | 13.07 ± 1.87 | 2.58 | 3.23 |
| Urea | 5.54 ± 0.35 | 21.20 ± 0.14 | 5.43 | 3.82 |
| (NH₄)₂HPO₄ | 3.09 ± 0.81 | 14.26 ± 0.52 | 2.44 | 4.61 |
| (NH₄)₂SO₄ | 2.39 ± 0.49 | 8.91 ± 0.58 | 2.23 | 3.73 |

*Values are given at the time of maximum EPS production. Data are means of three independent experiments ± standard deviation.

II. EPS production by Phoma sp. P98*

| Nitrogen | Biomass | Polysaccharide | | Specific production |
|---|---|---|---|---|
| source | (g/l) | (g/l) | PH | (g/g) |
| NaNO₃ | 11.46 ± 0.85 | 3.24 ± 0.63 | 7.22 | 0.28 |
| NH₄NO₃ | 6.12 ± 0.33 | 1.17 ± 0.43 | 2.33 | 0.19 |
| Urea | 8.09 ± 1.01 | 3.57 ± 0.97 | 6.18 | 0.44 |
| (NH₄)₂HPO₄ | 6.53 ± 0.44 | 0.00 ± 0.00 | 2.43 | 0.00 |

*Values are given at the time of maximum EPS production. Data are means of three independent experiments ± standard deviation.

Besides sodium nitrate, other nitrogen sources such as urea, ammonium nitrate, ammonium phosphate and ammonium sulphate were used. Remarkably, on urea, EPS production by Rhizoctonia sp. P82 and Phoma sp. P98 reached the same levels obtained on sodium nitrate.

### Example 4

### EPS Purification and Characterization

The EPSs produced by Rhizoctonia sp. P82, Phoma sp. P98 and Penicillium chermesinum P28 were purified. The polysaccharides were exclusively constituted of sugars, thus indicating suprisingly high levels of purity. Both thin layer chromatography (TLC) and gas chromatography (GC) analysis showed that the EPSs from Rhizoctonia sp. P82 and Phoma sp. P98 were constituted of glucose only. In contrast, that from P. chermesinum P28 was constituted of galactose with traces of glucose.

The molecular weights (MW) of the EPSs from Rhizoctonia sp. and Phoma sp., estimated by gel permeation chromatography using a 100×1 cm Sepharose CL4B gel (Sigma) column, were both approximately 2·10⁶ Da.

Determination of the position of the glucosidic linkages in the EPSs from Rhizoctonia sp. P82 and Phoma sp. P98 was carried out by GCms and GC after methylation, total hydrolysis, reduction and acetylation. The main products were identified by GCms analysis as glucitol 2,4-di-O-methyl-tetracetylated, glucitol 2,4,6-tri-O-methyl-triacetylated and glucitol 2,3,4,6-tetra-O-methyl-diacetylated indicating that both EPSs were characterised by monosaccharides linked with β-1,3 and β-1,6 linkages. In the case of the EPS from Phoma sp., the GC analyses showed three peaks in a quantitative ratio typical of a glucan with many branches; besides the above reaction products, the same type of analysis showed that the EPS from Rhizoctonia sp. gave rise to other reaction products such as penta- and esa-O-methyl-acetylated compounds which clearly indicated an uncompleted methylation.

Surprisingly, NMR analysis confirmed that both polysaccharides were pure, constituted of glucose only and characterized by β-1,3 and β-1,6 linkages.

### Example 5

### EPS Immuno-Stimulatory Effects

The EPSs from Rhizoctonia sp. P82 and Phoma sp. P98 were subjected to in vitro and in vivo experiments. A purified scleroglucan, obtained from S. glucanicum NRRL 3006, was used as a control. The purified EPSs were randomly broken in fragments of different molecular weights (from 1·10⁶ to 1·10⁴ Da) by sonication. The free glucose concentrations of the sonicated samples did not increase, thus indicating that no branches were broken. The experiments were carried out with EPSs at high MW (HMW, the native EPSs), medium MW (MMW, around 5·10⁵ Da) and low MW (LMW, around 5·10⁴ Da).

Immuno-stimulatory action was evaluated in vitro by determining effect on TNF-α production, phagocytosis induction, lymphocytes proliferation and IL-2 production.

All the EPSs stimulated monocytes to produce TNF-a factor; its content increased with increased polysaccharide concentration and was maximum when medium and low MWs were used.

In order to assess the effect of the EPSs on phagocytosis, two methods (Phagotest and Microfluoimetric Phagocytosis Assay) were used. The results gave a good indication that a high concentration of EPS improves phagocytosis.

In contrast, no significant effects were observed on lymphocyte proliferation and IL-2 production when the EPSs were added either alone or in combination with phytohemagglutinin (PHA). In addition, no cytotoxic effects were observed.

An in vivo study was carried out to assess immuno-stimulatory activity of the EPS using MMW (around 5·10⁵ Da) glucan from Rhizoctonia sp. P82.

Female mice were inoculated three times subcutaneously (SC) and/or orally (OR) with MMW EPS (2 mg/100 g weight) and Lactobacillus acidophilus (1·10⁸ cells/100 g weight) after 1, 8 and 28 days. Bleedings were carried out after 13 and 33 days. In vivo immuno-stimulation was evaluated by comparing antibody production by an ELISA test.

All the mice that received OR bacteria (groups 3, 4 and 5) showed no increase in their antibody content, regardless of their glucan inoculation. However, differences in antibody production were observed among mice inoculated SC with bacteria. Furthermore, antibody levels of mice that received SC only bacteria were significantly higher (P<0.01, by Tukey Test) than those that had received glucan and bacteria both SC and glucan OR and bacteria SC.

Interestingly, the results indicate that the EPS from Rhizoctonia sp. Gives rise to a decrease in antibody concentration. Remarkably, it can be concluded from this that the glucan from Rhizoctonia sp. causes activation of an antimicrobial activity of monocytes (see the effects described above relating to TNF-α production and phagocytosis induction) with a consequent reduction in the bacterial number leading, in turn, to a consistent reduction in antibody production.

In conclusion, the three filamentous fungi Rhizoctonia sp. P82, Phoma sp. P98 and Penicillium chermesinum P28 have a surprisingly good ability to produce extracellular polysaccharides of potential interest. In particular, Rhizoctonia sp. P82 is interesting in view of its short time required for fermentation, its high level of EPS production and its absence of β-glucanase activity during the EPS production phase. Furthermore, its EPS, as well as that from Phoma sp. P98, is a glucan characterised by β-1,3 and β-1,6 linkages. In addition, results relating to immuno-stimulatory effects of the glucan produced by Rhizoctonia sp. P82 indicate the possibility of a good stimulatory activity.

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications be covered by the appended claims.

What is claimed is:

1. A method for producing a beta-glucan which comprises fermenting a suspension comprising a non-pathogenic saprophytic filamentous fungus under conditions sufficient to produce a beta-glucan, and extracting the beta-glucan from the fermented suspension.

2. The method according to claim 1, wherein the non-pathogenic saprophytic filamentous fungus is selected from the group consisting of Penicillium chermesinum, Penicillium ochrochloron, Rhizoctonia sp., Phoma sp., or a combination thereof.

3. The method according to claim 1, wherein the non-pathogenic saprophytic filamentous fungi Penicillium chermesinum, Penicillium ochrochloron, Rhizoctonia sp. and Phoma sp. are fermented together to produce the beta-glucan in increased yield.

4. The method according to claim 1, wherein the fermenting is carried out for at least about 50 hours.

5. The method according to claim 1, wherein the fermenting is carried out in a medium comprising at least one component selected from the group consisting of NaNO₃, KH₂PO₄, MgSO₄, KCl and yeast extract.

6. The method according to claim 1, wherein the fermenting is carried out by cultivating the fungus in minimal medium which consists essentially of glucose and salts.

7. The method according to claim 1, wherein the fermenting is carried out by cultivating the fungus in a medium which comprises NaNO₃ (10 mM), KH₂PO₄ (1.5 g/l), MgSO₄ (0.5 g/l), KCl (0.5), C₄H₁₂N₂O₆ (10 mM) and glucose (60) and having a pH of 4.7.

8. The method according to claim 1, wherein the beta-glucan is added to a food product, a nutritional composition, or a medicament.

9. A method for enhancing one or more of structure, texture, or stability of a food product which comprises providing a beta-glucan by a non-pathogenic saprophytic filamentous fungus or composition containing same, and adding the beta-glucan to the food product in an amount effective to thereby enhance food structure, texture, stability or combinations thereof.

10. The method according to claim 9, wherein the non-pathogenic saprophytic filamentous fungus is selected from the group which consists of Penicillium chermesinum, Penicillium ochrochloron, Rhizoctonia sp., Phoma sp., or a combination thereof.

11. The method according to claim 9, wherein the fungus comprises a combination of Penicillium chermesinum, Penicillium ochrochloron, Rhizoctonia sp. and Phoma sp.

12. A method for providing nutrition in a food product which comprises providing a beta-glucan by a non-pathogenic saprophytic filamentous fungus or composition containing same, and adding the beta-glucan to the food product in an amount sufficient to increase its nutrition content.

13. The method according to claim 12, wherein the non-pathogenic saprophytic filamentous fungus is selected from the group which consists of Penicillium chermesinum, Penicillium ochrochloron, Rhizoctonia sp., Phoma sp., or a combination thereof.

14. The method according to claim 12, wherein the fungus comprises a combination of Penicillium chermesinum, Penicillium ochrochloron, Rhizoctonia sp. and Phoma sp.

15. A method for manufacturing a medicament or nutritional composition for the prevention or treatment of an immune disorder, tumor or microbial infection which comprises providing a beta-glucan by a non-pathogenic saprophytic filamentous fungus or composition containing same, and forming a medicament or nutritional composition from a therapeutically effective amount of the beta-glucan.

16. The method according to claim 15, wherein the non-pathogenic saprophytic filamentous fungus is selected from the group which consists of Penicillium chermesinum, Penicillium ochrochloron, Rhizoctonia sp., Phoma sp., or a combination thereof.

17. The method according to claim 15, wherein the fungus comprises a combination of Penicillium chermesinum, Penicillium ochrochloron, Rhizoctonia sp. and Phoma sp.

18. A method for enhancing one or more of structure, texture, or stability of a food product which comprises producing a beta-glucan by the method of claim 1, and adding the beta-glucan to the food product in an amount effective to thereby enhance food structure, texture, stability or combinations thereof.

19. A method for providing nutrition in a food product which comprises producing a beta-glucan by the method of claim 1, and adding the beta-glucan to the food product in an amount sufficient to increase its nutrition content.

20. A method for manufacturing a medicament or nutritional composition for the prevention or treatment of an immune disorder, tumor or microbial infection which comprises producing a beta-glucan by the method of claim 1, and forming a medicament or nutritional composition from a therapeutically effective amount of the beta-glucan.
